(19) United States
(12) Patent Application Publication
(10) Pub. No.: US 2021/0371804 A1
(43) Pub. Date: Dec. 2, 2021

(54) Videomicroscopy of contractile cell cultures and cell culture methods using same.

(71) Applicant: Institut de Cardiologie de Montréal, Montreal (CA)

(72) Inventors: Antoine Belzil, Montreal (CA); Philippe Comtois, Sainte-Therese (CA); Roselle Gelinas, Outremont (CA)

(21) Appl. No.: 17/334,952

(22) Filed: May 31, 2021

Related U.S. Application Data
(60) Provisional application No. 63/032,800, filed on Jun. 1, 2020.

(51) Int. Cl. C12M 1/34; C12N 5/077; C12N 5/071; G01N 33/50; C12M 3/00; G02B 21/36; G06T 7/20; G06T 7/00

(52) U.S. Cl. CPC C12M 41/46; C12N 5/0657; C12N 5/0697; G01N 33/5008; C12M 21/08; G06T 2207/10056; G06T 7/20; G06T 7/0016; G06T 2207/30024; G06T 2207/10016; G02B 21/365

(57) ABSTRACT

A method for characterizing contractions in cell cultures of contractile cells, the method comprising: acquiring a series of images of the contractile cells; for each image in the series of images, computing a movement index characterizing the rate of change in mean absolute pixel intensity variations across the whole image; and using the movement index to assess the contractions of the contractile cells. Also, applications of the method to the study of cardiac tissue analogs and other cell cultures, and a culture system used to implement the method.

### FIELD OF THE INVENTION

The present invention relates generally to the field of cell cultures, and more specifically to videomicroscopy of contractile cell cultures and cell culture methods using same.

### BACKGROUND

Cell culture of cardiac tissue analogs is useful for regenerative medicine (for example for conditioning of pre-injected stem cell-derived cardiomyocytes) and tissue engineering and widely used for high throughput cardiotoxicity evaluation. One important characteristic of such tissue analogs is their spontaneous or stimulated contractions. Some systems characterize this contraction using electrodes. Such systems are relatively expensive and by nature only provide relatively low resolution, limited by the number of electrodes and the inter-electrode spacing. Other systems use fluorescence microscopy for the same purpose. These systems are mechanically and optically complex and therefore relatively expensive. Also, the use of fluorescent markers is less than ideal as long exposition to such markers may be harmful to the cells that are studied, or at the very least interfere with their normal function, which may affect the effect one wishes to study.

Against this background, there exists a need for improved devices and method to study contractile cells in vitro. An object of the present invention is to provide such devices and methods.

### SUMMARY OF THE INVENTION

In a broad aspect there is provided a method for characterizing contractions in cell cultures of contractile cells, the method comprising: acquiring a series of images of the contractile cells; for each image in the series of images, computing a movement index characterizing the rate of change in mean absolute pixel intensity variations across the whole image; identifying peaks in the movement index; and using peak attributes of the peaks to assess the contractions of the contractile cells.

In another broad aspect, there is provided a method for assessing effects of a stimulus on cardiac cells, the method comprising: culturing a tissue sample including the cardiac cells; applying the stimulus to the cardiac cells; characterizing peaks obtained using the method for characterizing contractions defined above after application of the stimulus; and using characterization of the peaks to assess the effects of the stimulus on the cardiac cells. This method can be iterated to direct the application of stimuli conductive to achievement of predetermined tissue contraction properties, for example in a tissue engineering context. This method can also be used to assess the disruption to contractions caused by other stimuli, for example in a toxicology context.

Advantageously, the proposed method may be implemented efficiently using relatively inexpensive components. As the proposed method is non-invasive and non-disruptive, contraction assessments can be performed repeatedly over long periods of time without damaging the tissue under study. Also, in some embodiments, movement in the cultured tissue is characterized using a method that is very rapid to calculate, but that still provides useful information about the contractions of the cultures tissue, and consequently of its underlying physiological status.

In another broad aspect, there is provided a characterization method for characterizing contractions of a tissue in vitro, the tissue including cardiomyocytes, the method comprising capturing a sequence of images of at least part of the tissue over a predetermined time period, each of the images including a plurality of pixels, each pixel being characterized by a respective intensity value; and for each pair of images from the sequence of images separated by a predetermined delay, computing a movement index over a common region of the images, computing the movement index including summing over all the pixels contained in the region an absolute value of a difference between the intensity values of the pixels in the images of the pair of images, the movement indexes forming a sequence of movement indexes. The movement indexes are indicative of changes in sarcomere length in the cardiomyocytes contained in the region.

Typically, the camera remains fixed relative to a culture well in which the tissue is cultured so that any movement between images is due to intrinsic movements of the tissue. Also, the region of the images over which the movement index is computed may include the whole image or only a selected portion of the image. Also, after the sum over the whole region is taken, normalization, for example division by the number of pixels in the image and/or division by the time interval between successive images, may be optionally performed to obtain the movement index. In some embodiments, no normalization is performed.

There may also be provided a characterization method wherein characterizing the contractions of the tissue includes detecting peaks in the sequence of movement indexes.

There may also be provided a characterization method method wherein the peaks includes alternating major peaks and minor peaks, characterizing the contractions of the tissue includes calculating an amplitude ratio between major and minor peaks. Major peaks have a higher amplitude than minor peaks. In a contractile tissue, the major peak usually correspond to the relative rapid contraction of the tissue, while the minor peaks correspond to relaxation to an relaxed state. The amplitude ratio may be be computed between successive peaks and the averaged, or be calculated based on average amplitudes of the major and minor peaks, among other possibilities. If long sequences of images are obtained, and a stimulus is applied to the tissue while the sequence is acquired, a time evolution of the amplitude ration can be used to assess the physiological effect of the stimulus on the tissue as a function of time.

There may also be provided a characterization method wherein characterizing the contractions of the tissue includes calculating a peak delay between at least two of the peaks from the sequence of peaks. Delays between successive major peaks, successive minor peaks or between successive major and minor peaks could be assessed.

There may also be provided a characterization method further comprising culturing the tissue in a monolayer prior to capturing the sequence of images.

There may also be provided a characterization method wherein the movement index is a global movement index and the sequence of movement indexes is a global sequence of movement indexes, the method further comprising computing a local movement index in a sub-region of the region to obtain a local sequence of local movement indexes, characterizing the contractions of the tissue including comparing the local and global sequences of movement indexes.

There may also be provided a characterization method wherein comparing the local and global sequences of movement indexes includes computing a delay between peaks of the local sequence of movement indexes and peaks of the global sequence of movement indexes. In alternative embodiments, the local and global sequences of movement indexes are compared by cross-correlating them.

There may also be provided a characterization method wherein capturing the sequence of images is performed using lensless imaging.

In another broad aspect, there is provided an assessment method for assessing an effect of a stimulus on cardiomyocytes contraction, the cardiomyocytes being part of an in vitro tissue sample, the assessment method comprising: applying the stimulus to the in vitro tissue sample; obtaining a post-stimulus movement index sequence associated with the tissue after application of the stimulus using the method of claim 1; assessing the effect of the stimulus on the cardiomyocytes by comparing the post-stimulus movement index sequence to a pre-stimulus movement index sequence obtained either using the method of claim 1 on the in vitro tissue sample prior to application of the stimulus or obtained using a reference tissue sample similar to the in vitro tissue sample.

There may also be provided an assessment method wherein the stimulus is selected from the group consisting of pharmacological, chemical, physical, mechanical and electrical stimuli.

There may also be provided an assessment method wherein assessing the effect of the stimulus includes comparing contraction periods obtained from the post-stimulus movement index sequence and from the pre-stimulus movement index sequence.

There may also be provided an assessment method wherein assessing the effect of the stimulus includes comparing contraction amplitudes obtained from the post-stimulus movement index sequence and from the pre-stimulus movement index sequence.

There may also be provided an assessment method wherein assessing the effect of the stimulus includes comparing a variation in delay between successive contractions in the post-stimulus movement index sequence and in the pre-stimulus movement index sequence.

There may also be provided an assessment method wherein assessing the effect of the stimulus includes comparing root mean square values of the post-stimulus and pre-stimulus movement index sequences.

In another broad aspect, there is provided an apparatus for analyzing contraction of a tissue samples including cardiomyocytes on basis of a sequence of captured images, the apparatus comprising an image acquisition system configured to obtain the sequence of images depicting the tissue sample over an analysis period; and an analysis system configured to compute the movement index sequence as defined above.

There may also be provided an apparatus further comprising a culture chamber for containing the tissue sample.

There may also be provided an apparatus wherein the culture chamber is provided with a window, and the image acquisition portion is provided with an image sensor in register with the window and configured to perform lensless imaging of the tissue sample.

There may also be provided an apparatus further comprising at least one electrode for applying an electrical stimulus to the tissue sample.

In another broad aspect, there is provided a tissue culture method, comprising culturing a tissue including cardiomyocytes; and performing the method described above to obtain a sequence of movement indexes characterizing a physiological state of the tissue. In case the physiological state of the tissue meets a predetermined criteria, a stimulus is applied to the tissue, and otherwise, the tissue culture method is repeated.

Other objects, advantages and features of the present invention will become more apparent upon reading of the following non-restrictive description of some embodiments thereof, given by way of example only with reference to the accompanying drawings.

### BRIEF DESCRIPTION OF THE DRAWINGS

FIG. 1, in a flowchart, illustrates a characterization method for characterizing contractions of a tissue in vitro in accordance with an embodiment of the present invention;

FIG. 2, in a flowchart, illustrates an assessment method for assessing an effect of a stimulus on cardiomyocytes contraction in which the method of FIG. 1 is also used;

FIG. 3, in a schematic view, illustrates a cell culture system usable to perform the method of FIG. 1, the cell culture system also including components required to perform the method of FIG. 2;

FIG. 4 illustrates examples of a movement index ΔS obtained using an embodiment of the method of FIG. 1 with different delay between frames. Panel A) τ=1 frame, Panel B) τ=4 frames, and Panel C) τ=8 frames. The video was recorded at 50 fps.

FIG. 5 illustrates stimulation of contraction and the link to the movement index ΔS. Panel A) Shortening of the sarcomere length (SL) as a function of time using the contraction model only (uncoupled from the neonatal cardiomyocyte ionic model) using a simulated calcium transient with amplitude of 1.45 μmol/L. Panel B) The time derivative of SL with a Δt=0.01 ms. The dots correspond to the derivative calculated with a sub-sample signal corresponding to 50 fps. Panel C) Rectified signal of panel B showing double peaks corresponding to the contraction and relaxation phases. Panel D) Explanation of two specific measures (Δ_{R-C} and Ratio_{C/R}) obtained from two different calcium amplitudes (1.0 μmol/L and 1.45 μmol/L). A lower [Ca²⁺] amplitude leads to a decreased Δ_{R-C} and increased Ratio_{C/R}.

FIG. 6 illustrates classification of ΔS peaks. Panel A) An example of ΔS with detected peaks (red circles) showing alternation between high amplitude (major) and low amplitude (minor) peaks. Inset: blow up of the activity within the dashed black rectangle which highlights the high amplitude contraction peaks followed by the lower amplitude relaxation peak similar to the derivative of the simulated contraction model. Panel B) Contraction and relaxation clusters are relatively well separated when viewed in the space with x-axis being the time between markers and y-axis being the peak amplitude (ΔSₚₑₐₖₛ). Panel C) Estimation of the change in ΔS around the peaks given by the Ratio_{drop} (amplitude of 1^{st} and 2^{nd} samples around the peaks divided by the peak amplitude). Lower ratio Ratio_{drop} is linked to faster change in ΔS around the peaks for the contraction peaks compared to the relaxation.

FIG. 7 illustrates temporal variation in activity obtained by analysis of ΔS with 50 fps. Panel A) The signal obtained for a representative sample (blue line). Detected peaks are highlighted by red circles. Post classification results are shown by dotted red lines (contraction peaks) and dashed red lines (relaxation peaks). Panel B) The period of activity given the time difference between contraction peaks shows a constant period of activity. Panel C) Calculated Δ_{R-C} as a function of the period of activity (corresponding here to the time elapse since the last contraction) is only varying by ±one sample. Panel D) Ratio_{C/R} is more varying compared to Δ_{R-C}.

FIG. 8 illustrates experimental data obtained for control samples. Panel A) Mean temporal Δ_{R-C} (<Δ_{R-C}>) is increasing when the mean T (<T>) is augmenting. The red line corresponds to the linear regression fit (slope of 0.072, p<0.001, corr.=0.89). B) Mean Ratio_{C/R} (<Ratio_{C/R}>) as a function of <T> showing a slight augmentation with increasing <T>. The linear regression line (red line) has a slope of 0.276 (p<0.01, corr=0.56).

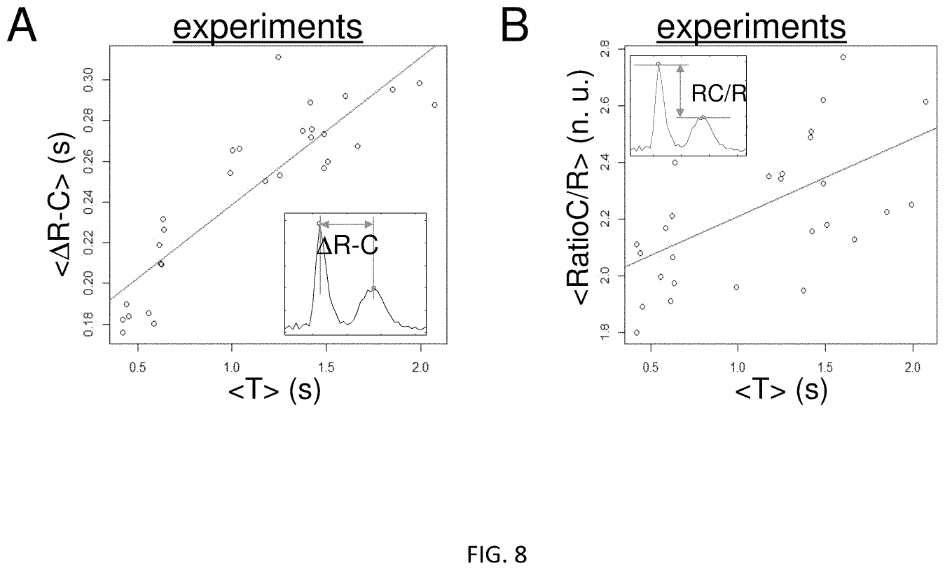

FIG. 9 illustrates results similar to those of FIG. 8, but for simulations Panel A) Increasing <Δ_{R-C}> with augmentation of <T>. Panel B) Decreasing <RatioC/R> with increasing <T> which is the inverse of the experimental result.

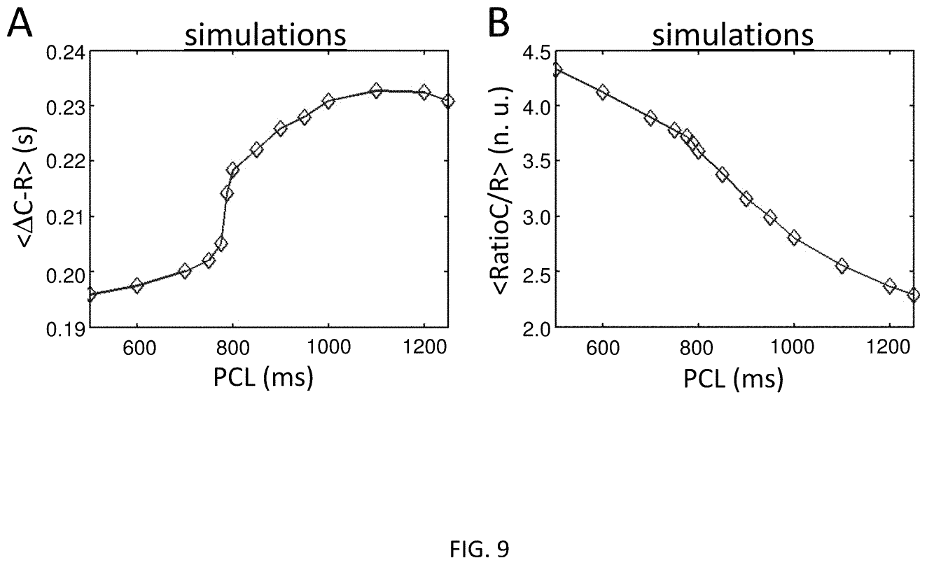

FIG. 10 illustrates comparison between CTL (black points) and ISO (white circles) contraction results. Panel A)<Δ_{R/C}> as a function of <T> does not shows dissimilarity between groups. Panel B)<Ratio_{C/R}> as a function of <T> differs between CTL and ISO with both a significant <T> and group effects.

FIG. 11 Illustrates estimation of heterogeneous contraction rhythms within the FOV. Panel A) Map of log(E) with the energy E calculated as described in Example 1, showing area with no contraction signal in blue. Panel B) Histogram of log(E) with the dashed line representing the threshold (log(E)>6) use to select high energy signal regions. Panel C) Maximum correlation coefficient and Panel D) lag between local ΔS_{x′,y′}(t) and global ΔS(t) for regions having log(E)>6. Two low CC regions labelled sub 1 and sub 2 are highlighted. Most of the regions show high CC with 0 s lag. Panel E) Signal from region sub 1 (top axes) and sub 2 (bottom axes) showing the low CC (shown as red lines vs. global ΔS as dotted blue lines) and global ΔS.

FIG. 12 illustrates analysis of a complex global ΔS. Panel A) Example of a non-stationary global signal with time intervals where multiple peaks (different to the contraction/relaxation peaks usually found). Two intervals are highlighted by dashed rectangles with the orange one showing the normal double peak signal and the red rectangle with multiple peaks. Panel Bi) Map of log(E) after thresholding (log(E)>4). Panel Bii) cross-correlation (CC) CC between individual cluster signals and global ΔS. Biii) Labelled separated clusters with colorscale representing the CC between cluster 5 and other clusters. Panel C) Matrix of CC between clusters (1-5) with diagonal equal to 1 and D) lag for maximum CC. Minimum CC and maximum lag is found between cluster 5 and all 4 other clusters. Panel E) Cluster ΔS signals for the 5 within the two time intervals highlighted by rectangles in panel A is shown by a grayscale (peaks are darker and lower values whiter). The global ΔS is plotted in blue on top for comparison;

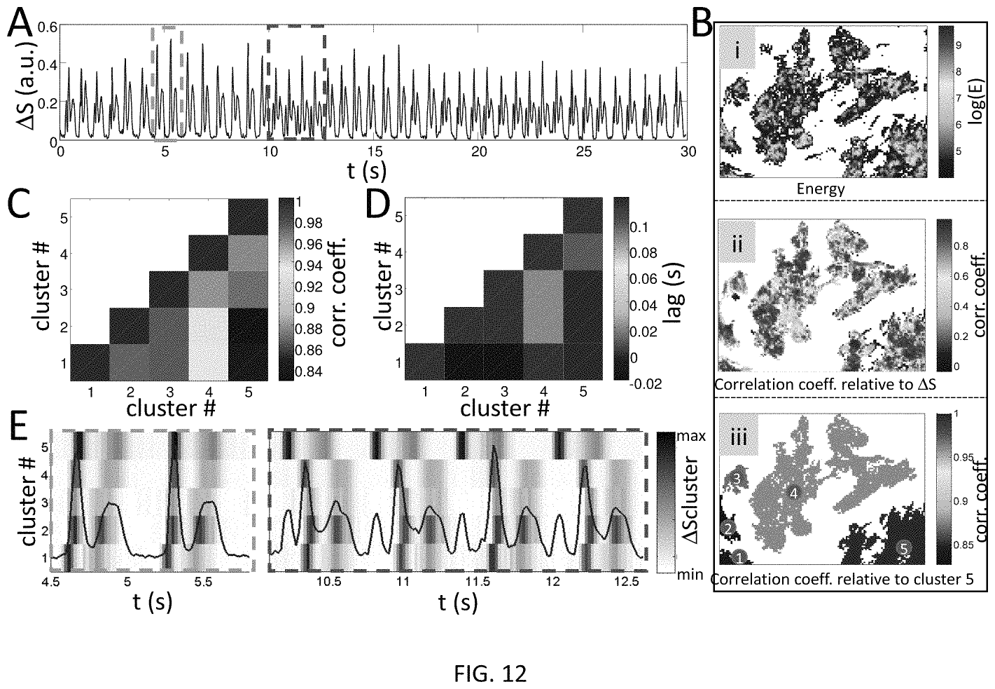

FIG. 13 illustrates in block diagram form the physical and software structure of a specific implementation of the culture system of FIG. 3. Panel A: Control of the various sub-systems of the culture system. Panel B: image acquisition architecture. Panel C: GStreamer pipeline used to provide real time display; and

FIG. 14 illustrates images acquired with the system of FIG. 13;

FIG. 15 illustrates global contraction of cell cultures determined using the system of FIG. 13; and

FIG. 16 illustrates the effect of electrical stimulation on cell cultures using the system of FIG. 13.

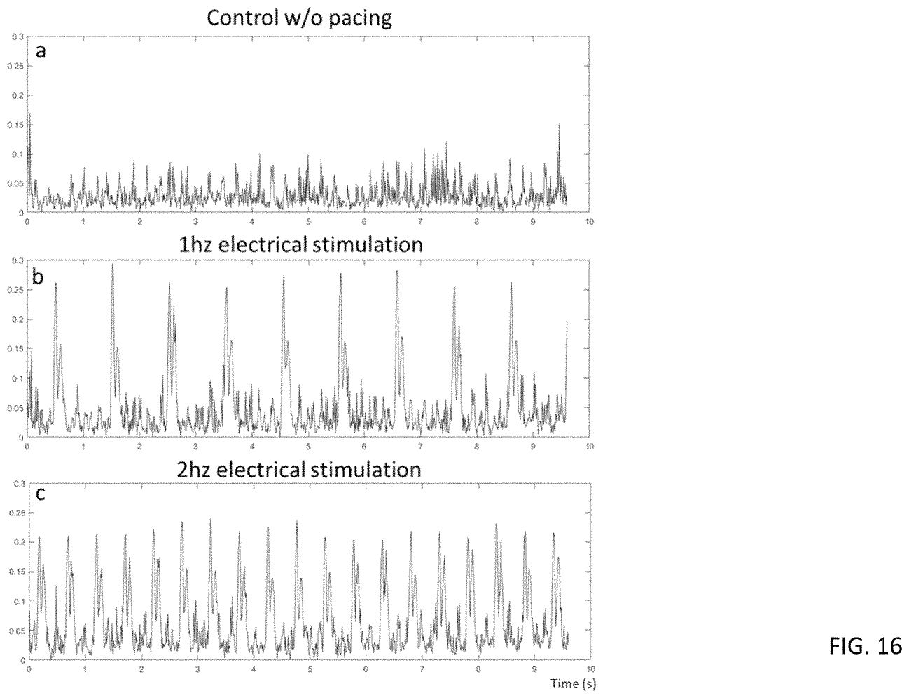

### DETAILED DESCRIPTION

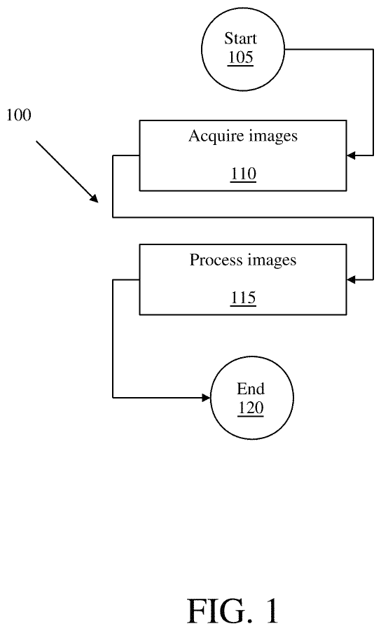

FIG. 1 is a flowchart of a method 100 for characterizing contractions in cell cultures of contractile cells, typically in vitro, in a culture. The method is then performed using a cell culture system, for example using a cell culture system 300 described in further details below. The method 100 starts at step 105. Then, at step 110, a sequence of images of the contractile cells to study is acquired at successive times. Typically, the images are acquired at a constant rate, for example between 100 and 300 frames per second (FPS). However, if time stamps are used, images may be acquired with variable time separation between successive imaged. The images each include a plurality of pixels, each pixel being characterized by a respective intensity value. The pixels may also be characterized by more than one intensity value (in case of a multi-channel image acquisition system), or by an intensity value and by other characteristics indicative of color, such as hue).

Once enough images have been acquired, step 115 of processing the images is performed. The minimal number of images depends on the delay between images used to compute a difference indicative of changes in the images, as described in further details below, though a movement index. In a specific example, step 115 includes computing for each image in the series of images the movement index characterizing the rate of change in mean absolute pixel intensity variations across a region of the image or across the whole image. In a very specific example, step 115 includes identifying peaks in the movement index and using peak attributes to assess the contractions of the contractile cells. The method then ends at step 120. The proposed method may be used to characterize spontaneous contractions in cultured cardiac tissue and similar tissues, or stimulated contraction, for example using pharmacological, chemical (by varying ionic concentrations for example), mechanical or electrical stimulation. In some embodiments, the movement index is indicative of changes in sarcomere length in the cardiomyocytes contained in the region. Characterization may be performed for example by displaying the movement index as a function of time, or by calculating a metric associated with the movement index and displaying or otherwise conveying this metric to an intended user. In other embodiments, the metric is is fed to an automated software the takes predetermined actions when the metric is within predetermined intervals.

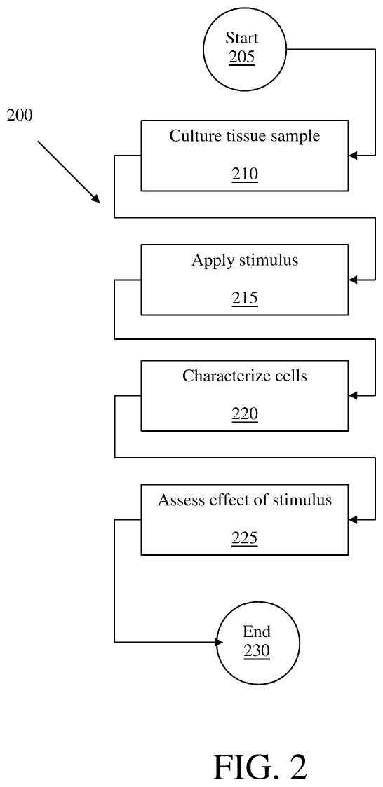

FIG. 2 is a flowchart of an application of the method of FIG. 1. FIG. 2 illustrates a method 200 for assessing effects of a stimulus on cardiac cells (typically the cardiomyocytes). Method 200 starts at step 205. The method includes a step 210 of culturing a tissue sample including the cardiac cells. Then, at step 215, a stimulus is applied to the cardiac cells. The stimulus may for example, and non-limitingly, be electrical, mechanical or pharmaceutical. Subsequently, at step 220, the method 100 is used to characterize the cardiac cells. In other words, then the effect of the stimulus on the cardiomyocytes is assessed by, for example, comparing a post-stimulus movement index sequence to a pre-stimulus movement index sequence. For example, the peaks of the movement index of the example of step 110 given in the previous paragraph are characterized, such characterization being an example of a metric mentioned hereinabove. Examples of characterization includes assessing one or more of the width, amplitude and timing separation of the peaks. Then, at step 225, characterization of the cells is used to assess the effects of the stimulus on the cardiac cells. For example the observed peaks of the movement index can be compared to peaks obtained in a control experiment, performed separately from the experiment in which the stimulus has been applied, or peaks obtained from the same sample, but before application of the stimulus. Finally, the method ends at step 230.

Method 200 may be used in many contexts. In a first example, the proposed method 200 may be used to assess the toxicity of various compounds on cardiac cells, or the effect of any stimulus on the cardiac cells. Indeed, such toxicity will often result in changes in contraction characteristics, such as for example contraction periods or speeds (assessed for example by peak values of movement index or widths of the peaks). In other applications, the proposed method 200 may be used iteratively to create cultures tissues having predetermined properties. Indeed, one may stimulate a cell culture until a predetermined contraction characteristic is achieved by periodically monitoring the effect of the stimulation on the cardiac tissue in culture, or by modifying culture parameters and stimulation once the cultured cells show predetermined contraction characteristics. Other stimuli, such as pharmacological, chemical or physical (for example temperature, gas concentrations) could also be applied. Many other applications of the proposed method are also possible. Also, the present method could be applied to the study of cultures including other types of contractile cells, such as smooth or skeletal muscle cells. The samples studied may include essentially only the contractile cells, or other cell types may be provided along the contractile cells, such as connective tissue cells, among other possibilities.

In the examples below, monolayers of cells have been investigated. However, cell cultures including thin leaflets that are more than one cell thick could also be studied using the methods and devices presented herein.

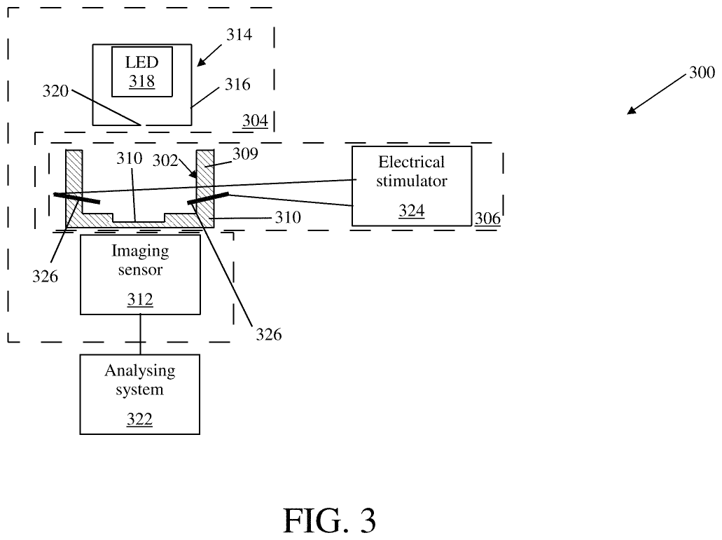

FIG. 3 illustrates an example of a cell culture system 300 usable to perform the methods of FIGS. 1 and 2. While the cell culture system 300 is well-suited for that purpose, alternative cell culture systems could also be used in alternative embodiments of the invention. The system 300 includes a culture well 302 and an image acquisition system 304. In some embodiments, the system 300 also includes an electrical stimulation system 306. Although not shown in the drawings, the culture system 300 could also include a mechanical stimulation system operative for either directly deforming a tissue cultured in the culture well 302, or for deforming the culture well 302 in embodiments in which the tissue cultured in the culture well 302 adheres to the culture well 302 sufficiently to allow mechanical stimulation in this manner.

The culture well 302 may have any suitable shape, such as, for example, a generally cylindrical shape. The culture well 302 typically defines a well bottom wall 308 and a well peripheral wall 309 extending therefrom. The well bottom wall 308 typically defines a window 310. The window 310 is a portion of the well bottom wall 308 that is transparent and thinner than the remainder of the well bottom wall 308. The window 310 is also typically substantially planar. The window 310 is typically thin enough to allow acquisition of images of the tissue that is cultured through the window 310, for example and non-limitingly, using lensless imaging techniques, in which images are acquired directly using an image sensor, without lens focusing. Since such imaging techniques perform better in cell cultures when the cultured tissue is closer to the image sensor, the window 310 should typically be as thin as possible while preserving mechanical integrity of the well bottom wall 308.

In some embodiments, the culture well 302 is integrally formed as a single piece of material using a transparent silicone material, such as Polydimethylsiloxane (PDMS). Such materials are advantageous as they present good mechanical properties suitable for cardiac cell cultures while being transparent, which allow manufacturing of the culture well 302 in a single piece of material, without requiring attachment of a separate window material, as would be needed is an opaque material were used for the remainder of the culture well 302.

The image acquisition system 304 includes an image sensor 312 and a light source 314. For example, the light source 314 takes the form of an enclosure 316 in which a light emitting diode (LED) 318 is positioned. A pinhole 320 facing generally towards the image sensor 312 allows light produced by the LED 318 to illuminate the tissue sample to analyze, which is contained in the culture well 302, the latter being positioned so that the window 310 is between the light source 314 and the image sensor 312. However, any other light source suitable for the type of imaging performed could be used.

The image sensor 312 is for example a CMOS sensor. It should be noted that the image sensor 312 is shown separated from the window 310 in FIG. 3, but that this is for facilitate illustration at the schematic level. In practice, it is advantageous to have the image sensor 312 and the window 310 as close to each other as possible, with the image sensor 312 touching the window 310 in some embodiments.

The image sensor 312 is connected to an image analyzing system 322, which acquires and analyzes the data provided by the image sensor 312. The image analyzing system 322 may include an embedded system that can provide processed data, a general purpose computer running suitable software, or both an embedded system and a general purpose computer.

When present, the electrical stimulation system 306 includes an electrical stimulator 324 operable to provide electrical signals having predetermined characteristics. The electrical stimulator 324 is electrically coupled to electrodes 326 positioned in the culture well 302, for example a pair of electrodes 326 opposed to each other with the window 310 therebetween. It should be noted that only one electrode 326 or more than 2 electrodes 326 could be used in alternative embodiments of the invention, depending on the type of electrical stimulation to provide. The electrodes 326 are able to transmit electrical signals having predetermined current or voltage characteristics across a culture medium contained in the culture well 302 to electrically stimulate the cardiac cells cultured in the culture well 302. The electrical stimulation system 306 may be used to electrically stimulate the cardiac cells to produce an effect while the cardiac cells are cultured, or could be used to assess directly the effect of an electrical signal applied on the cardiac cells on contraction properties of these cells. In other words, the electrical signals may be used to produce changes in the cardiac cells, or as a diagnostic tool to study the reaction of the cardiac cells to electrical stimulation.

Step 115 of processing the images is now described in further details. While many different processing techniques could be used, the techniques described hereinbelow have been found to be well-suited to the proposed culture system 300 as they are relatively straightforward to implement and can be performed in some embodiments quickly enough to provide real-time processing with relatively inexpensive equipment. Generally speaking, the techniques aim at conveying the large scale contraction characteristics of the tissue samples under investigation. Such techniques may consider a sample as a whole, or split each sample into a grid of sample regions, and examine how these regions relate to each other.

In a first example, whole sample images are used to assess contractions of a cardiac tissue in culture. Such tissues can show spontaneous contractions once the cells have grown to a point where they are adjacent to each other, with little or no uncolonized regions in the tissue, and once the cells have reached sufficient maturity. Let M(t) be the global camera frame recorded at time t, its pixels characterized by intensity values denoted by M_{i,j}. The movement index, also referred to as a composite signal, ΔS is calculated using the following equation:

$$Δ S ( t ) = \frac{1}{{N}_{x} {N}_{y}} {\sum }_{j = 1}^{{N}_{y}} {\sum }_{i = 1}^{{N}_{x}} \vert \frac{{M}_{i , j} ( t ) - {M}_{i , j} ( t - τ )}{τ} \vert$$

where τ is a discrete delay (or interval between frames), N_{y} and Nₓ are respectively the number of horizontal and vertical pixels. ΔS averages the speed of absolute variation in the pixel intensity over the whole image. It has been found that this global measure of image changes provides adequate information to characterize contraction and relaxation of periodically contracting in vivo cardiac tissue monolayers.

With synchronized tissues, contraction and relaxation can be seen as alternating peaks in the ΔS signal. One contraction peak is followed by a relaxation peak, which is itself followed by the next contraction peak. Delay between successive contraction and relaxation peaks are similar to the QT interval in whole hearts.

In other examples, one may wish to divide the whole images in regions and use the equation provided hereinabove to compute ΔS over each region. Then, correlations between ΔS of each region between the regions of with the global ΔS may be used to estimate the degree to which the image field contracts as a single entity or many entities. Correlations between the various regions may also be used to delimitate domains having similar contraction synchronization using well-known segmentation algorithms.

The proposed culture system 300 could also be used to directly analyze the images that are provided to extract characteristics of the cell in culture. Conventional image processing algorithms or neural networks could be used for that purpose. In addition, the culture system 300 could be used to assess cell migration, in addition to cell contraction.

### EXAMPLE

Cell culture of cardiac tissue analog is interesting for regenerative medicine (cell therapy and tissue engineering) and is widely used for high throughput cardiotoxicity. As a cost-effective approach to rapidly discard new compounds with high toxicity risks, cardiotoxicity evaluation is firstly done in vitro requiring cells/tissue with physiological/pathological characteristics (close to in vivo properties). Studying multicellular electrophysiological and contractile properties is needed to assess drug effects and techniques favoring process automation which can help in simplifying screening drug candidates. In this example, we present new approaches that use the videomicroscopy video of monolayer activity to study contractile properties of beating cells in culture. Two variables are proposed which are linked to the contraction dynamics and are dependent on the rhythm of activity. Methods for evaluation of regional synchronicity within the image field of view are also presented that can rapidly determine regions with abnormal activity or heterogeneity in contraction dynamics.

All animal-handling procedures were concordant with the Canadian Council on Animal Care guidelines and were approved by the institutional Animal Research Ethics Committee. Isolation was performed according to the protocol of the neonatal cardiomyocyte isolation kit from Worthington. In summary, one to three days old rats (Sprague-Dawley, Charles River) were sacrificed by decapitation. Beating heart were removed from the rats and immediately put in cold Ca²⁺ and Mg²⁺-free Hank's Balanced Salt Solution. Ventricular muscle was selected by excision and tissue was minced on ice into 1-3 mm³ pieces. The mixture was subjected to purified enzymatic digestion (trypsin and collagenase). Isolated cells (enriched cardiomyocytes) were counted and seeded at a density of 10⁶ cells/mL in the seeding area of the membrane pre-coated with 0.2% porcine-derived gelatin (G1890, Sigma) and 0.00125% fibronectin solution (F1141, Sigma). Cells were grown for 24 hours in DMEM (319-050-CL, Wisent) with 5% fetal bovine serum (FBS, SH30396.03, Fisher Scientic Co. Ltd) and 1% penicillin/streptomycin (450-201-EL, Wisent). Cardiomyocytes were then FBS starved with 1% penicillin/streptomycin in DMEM 24 h prior to the experiments.

Acute effects of the β-adrenergic agonist, isoproterenol (ISO, 16504, Sigma-Aldrich) was studied by videomicroscopy with final concentration of 100 nM at 1 minute after injection.

Phase contrast videos of neonatal cardiomyocytes were acquired after 48 hours post seeding in vitro with a Dalsa HM640 camera (N_{y}=640×Nₓ=480 pixels) at rates of 30, 50, or 100 fps coupled to an inverted Nikon optical microscope (10× magnification). The field of view (FOV) covered by the camera was 0.44 mm by 0.33 mm.

A neonatal rat ventricular myocyte (NVRM) mathematical ionic model (32) has been modified. The original spatial discretization implemented to study the effect of hypertrophy on calcium handling has been replaced by the common-pool calcium handling of the mice neonatal cardiomyocyte model (33). The pacemaker current (Iₜ) activation and time constant dynamics have been fitted based on published data (34). Detailed changes can be found in the supplemental table. The maximum conductance of Iₜ has also been change from 0.021 mS/μF to 0.06 mS/μF (34). The potassium transient outward current (Iₜₒ) activation and inactivation have been modified to fit the data published by Wickenden et al (35). The modified rat neonatal ventricular cardiomyocyte ionic model has been coupled to the Rice et al (36) cardiac myofilament model for contraction simulation. The complete model was numerically integrated in Matlab (R2008, MathWorks Inc., Natick, Mass.) using a simple Euler integration scheme with a constant 20 μs time step.

All statistical analyses have been done in R (version 3.1.3). Linear regression was used to test for variable dependency on period of activity. The analysis of covariance (ANCOVA) was used to compare group and covariate effects. Means were compared with Students t-test.

The proposed initial approach is using the raw video signal and calculating for each pixel the difference between frames separated by a delay corresponding to a defined frame interval, using the formula for ΔS mentioned hereinabove.

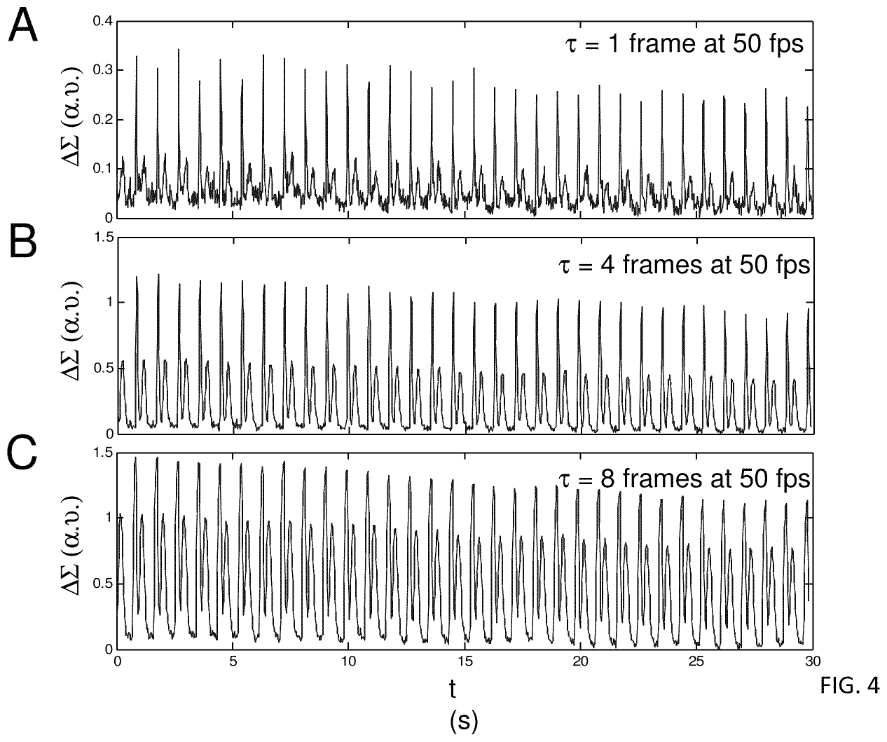

An example of the composite signal ΔS is shown in FIG. 4 for different delay (Σ=1,4, and 8). The shortest delay τ=1 corresponding to a frame by frame difference shows the highest noise level (panel A) while increasing τ to 8 decreased the amplitude difference between the high and low amplitude peaks (panel C).

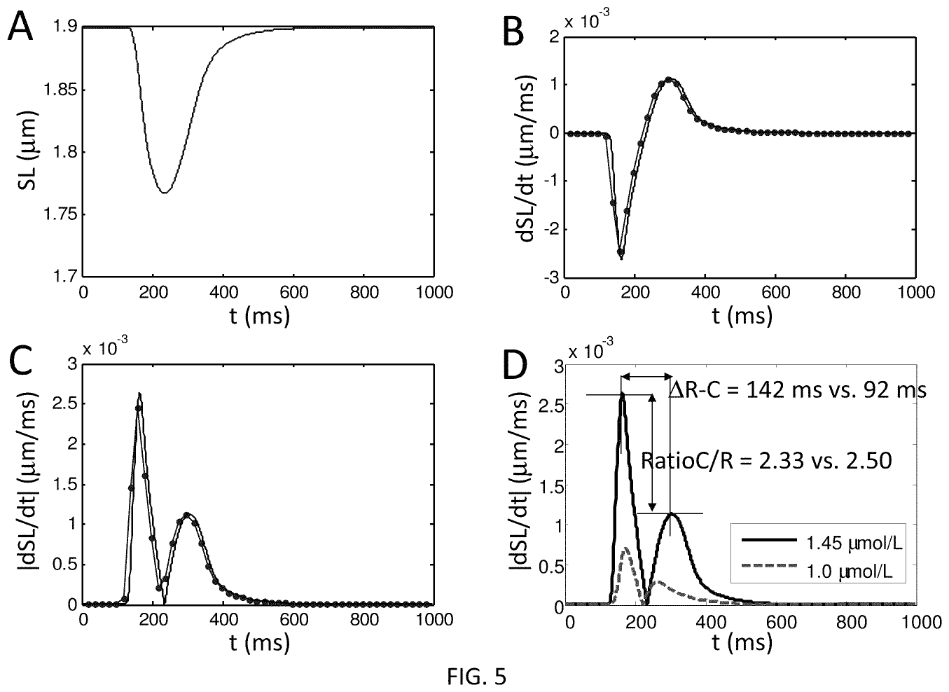

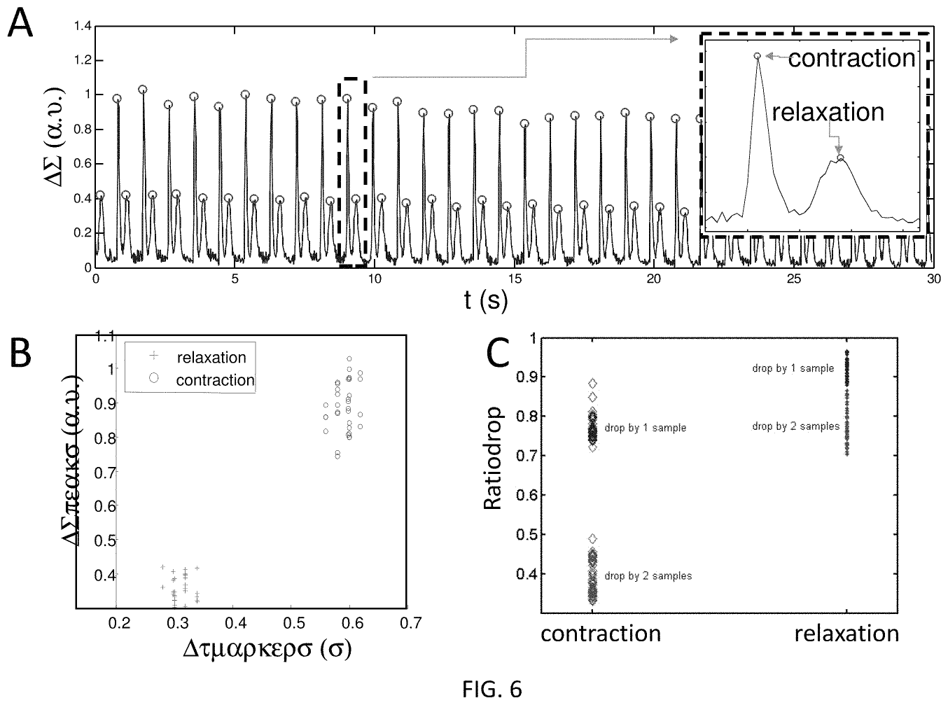

In silico data and analysis reveal that the composite signal ΔS can be interpreted as follow. The absolute derivative of the time-dependent cell length calculated from simulated sarcomere length (SL) obtained from the simulation is given by

$${dSL}_{abs} ( t ) = \vert \frac{dSL}{dt} \vert$$

where SL is the sarcomere length obtained by simulating the Rice et al model (36). The change in SL calculated with the contraction model only is presented in FIG. 5, panel A. The time derivative of this contraction signal is depicted in FIG. 5, panel B which shows the initial fast contraction (negative derivative) followed by the slower positive relaxation signal. As shown in FIG. 5 panel C, there is a clear similarity between the rectified derivative given and the videomicroscopy signal as shown in FIG. 4, panel A and FIG. 6, panel A.

Two specific measures are proposed that are presented in FIG. 5, panel D which corresponds to the amplitude ratio between the contraction and relaxation peaks (Ratio_{C/R}) and the time between the contraction and relaxation peaks (Δ_{R-C}). The effects of the calcium transient amplitude on contraction signal and specific measures is shown in FIG. 2D which highlights the sensitivity of the measures on calcium transient changes. Two different calcium concentration amplitudes were simulated corresponding to 1.0 and 1.45 μmol/l resulting to respectively the lowest and highest signal. A decrease in maximum [Ca²+] from 1.45 to 1.0 μmol/l yielded an increase of 7% in Ratio_{C/R} but a decrease of 54% in Δ_{R-C}.

Automatic discrimination between contraction and relaxation peaks is advantageous in the perspective of user-friendliness and for the approach to have a clear potential in a high throughput screening/testing system. In a specific embodiment, three conditions are being used to detect and validate classification of the peaks: 1—there is alternance between contraction and relaxation peaks, 2—clusters are usually separated in the variable space given by the time difference between peaks (Δtₘₐᵣₖₑᵣ) and amplitude of the peaks (ΔSₚₑₐₖₛ) as depicted in FIG. 6, panel B, 3—sharpest peaks are found for contraction and widest peaks for relaxation resulting in greater amplitude loss around the maximum ΔS peak amplitude. The last condition can be easily evaluated by taking the amplitude for 1 sample and 2 samples around the maximum amplitude of the peak divided by the peak amplitude (Ratio_{drop}). The obtained data are presented in panel C where a lower average Ratio_{drop} is found for the contraction peaks (left circles; 0.77±0.03 n.u. and 0.40±0.06 n.u.) compared to the relaxation peaks (right dots; 0.92±0.02 n.u. and 0.78±0.04 n.u.).

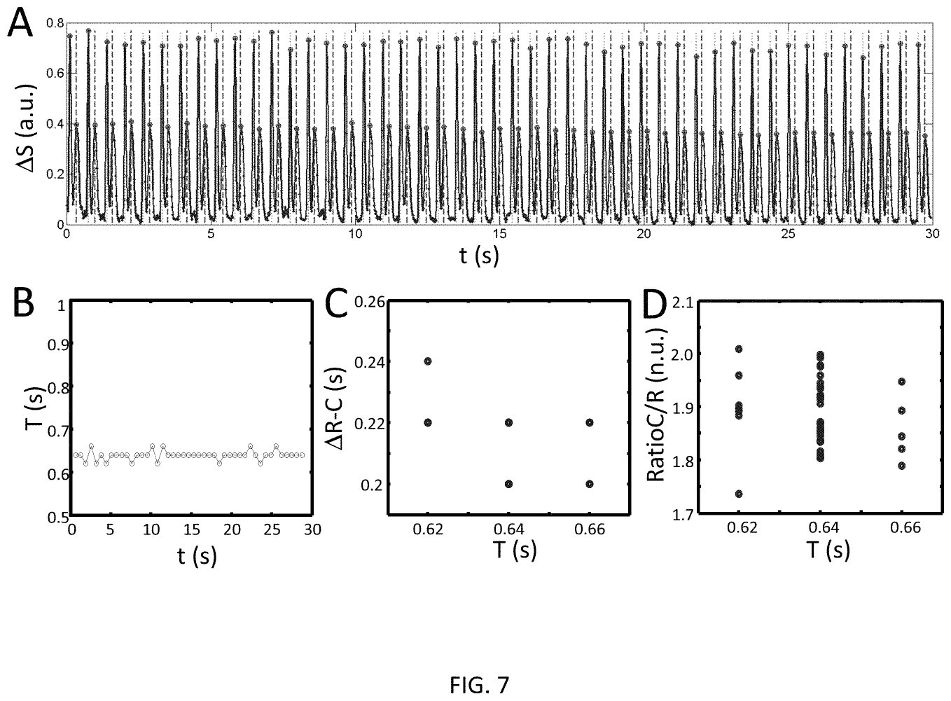

An example of an analyzed acquisition is presented in FIG. 7. The signal ΔS is shown in panel A with detected peaks (circles) and resulting classification highlighted for contraction (dotted lines) and relaxation peaks (dashed lines). The resulting series of activity shows a constant period T (panel B: 0.64±0.01 s) and almost no variation in Δ_{R-C}(panel C: 0.22±0.01 s). However, Ratio_{C/R} shows greater variability between samples (panel C: 1.89±0.06 n.u.) mainly due to variations in the maximum amplitude of the contraction peaks as seen on the signal in panel A.

A set of samples (n=29) has been analyzed. For each video of 30 s duration, the average values <T>, <Δ_{R-C}>, and <Ratio_{C/R}> were calculated as the average of the temporal values obtained from the signal analysis of individual acquisition. A clear monotone increase in <Δ_{R-C}> with increasing <T> is found as depicted in FIG. 6A with ˜40% change between the minimum and maximum values. The linear regression (p<0.001) has a slope of 0.072 s/s (represented as a red line) and intercept of 0.166 s. As expected, the variability is greater for <Ratio_{C/R}> but a trend to an increasing ratio as <T> augments is found. However, the variation with the period is less with ˜23% change between the minimum and maximum values in the dataset (see FIG. 6B). The fitted regression line (p<0.005, red line on the panel) has a slope of 0.276 s⁻¹ and an intercept of 1.93.

Steady-state simulations of the combined neonatal rat cardiomyocyte ionic model coupled to the contraction model have been done for a comparison purpose with the experimental data. Similar to experimental data, <Δ_{R-C}> augments with <T> increasing. However, the range of variation is slightly lower than in experiments. The <Ratio_{C/R}> has the inverse behavior compared to experimental data shown in FIG. 6B with decreasing ratio for increasing <T> and a over wider range. The causes of this difference remain to be elucidated.

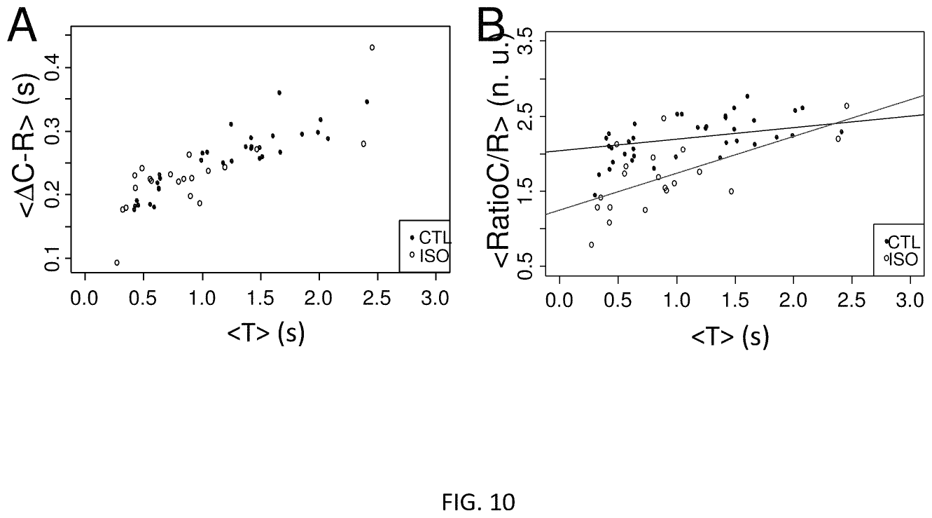

The variation in the contraction measures that can be evaluated by our videomicroscopy approach has been tested with isoproterenol (ISO), a β-adrenergic agonist. Results are presented in FIG. 10. As expected, the period of activity is significantly decreased by ISO compared to CTL (0.9±0.6 s vs. 1.9±2.4 s in CTL, p<0.05). Similar variation of <Δ_{R-C}> is found between CTL and ISO groups as a function of <T> although <T> is in average smaller with ISO as expected (panel A). Interestingly the <Ratio_{C/R}> showed statistically significant <T> and group effects with p<0.001. The slope of the linear regression being larger for the ISO group compared to CTL (0.146 for CTL vs. 0.490 for ISO).

All the previous analyses presented in this study are based on a global composite signal calculated using the entire Field of view (FOV). The same approach can be used for sub-regions of the FOV by calculating a composite signal for each section. We present here two additional approaches aiming to study spatial-temporal differences in videomicroscopy signals.

The first approach is based on determining how local signal correlates with the global composite signal. The local composite signals are calculated over sub-regions of N_{sub,x} and N_{sub,y} pixels from the total FOV using the formula for ΔS, but applied to regions.

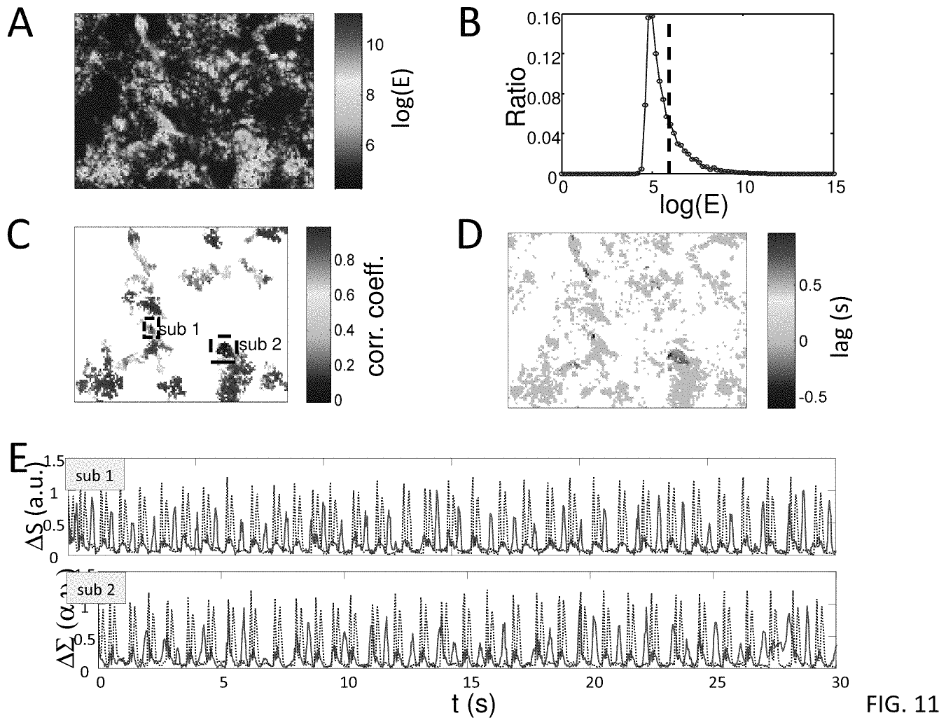

The energy (E) of the local composite signals are calculated using

$${E}_{{x}^{′} , {y}^{′}} = \frac{1}{{N}_{t} - τ} {\sum }^{{N}_{t}} \text{?} {( \frac{d Δ {S}_{{x}^{′} , {y}^{′}} ( t )}{dt} )}^{2}$$

$$\text{?} \text{indicates text missing or illegible when filed}$$

where Nₜ is the number of frames of the original video and the indices i and j index each subframe. An example of the spatial distribution of log(E_{x′,y′}) is displayed in FIG. 11, panel A and the corresponding histogram can be found in panel B. High energy regions of the FOV are selected using a thresholding approach.

The correlation coefficient (CC) and lag between the global signal ΔS(t) and local composite signals ΔS_{x′,y′}(t) are calculated. An example of the resulting map of coefficients and lag are respectively depicted in FIG. 11 panels C and D after keeping pixels with log(E)>6. In this example, most of the relevant section of the FOV have a correlation coefficient greater than 0.8 and a lag of 0 sec which indicates that the local activity is highly similar between these regions and the global activity. However, some regions show lower correlation including regions with a correlation value of less than 0.4 (regions labelled sub 1 and 2 in panel C). Interestingly, these regions have also non-zero lags. Using a thresholding approach on the correlation coefficient map, two corresponding clusters of low correlation with high energy can be detected and the average signal from these clusters are shown in panel E. The signals in both sub 1 and 2 regions (full line) have strong peaks usually not occurring simultaneously with the global composite signal (dotted line). Please note that lower amplitude peaks are also found in this signals that correlate with the global activity.

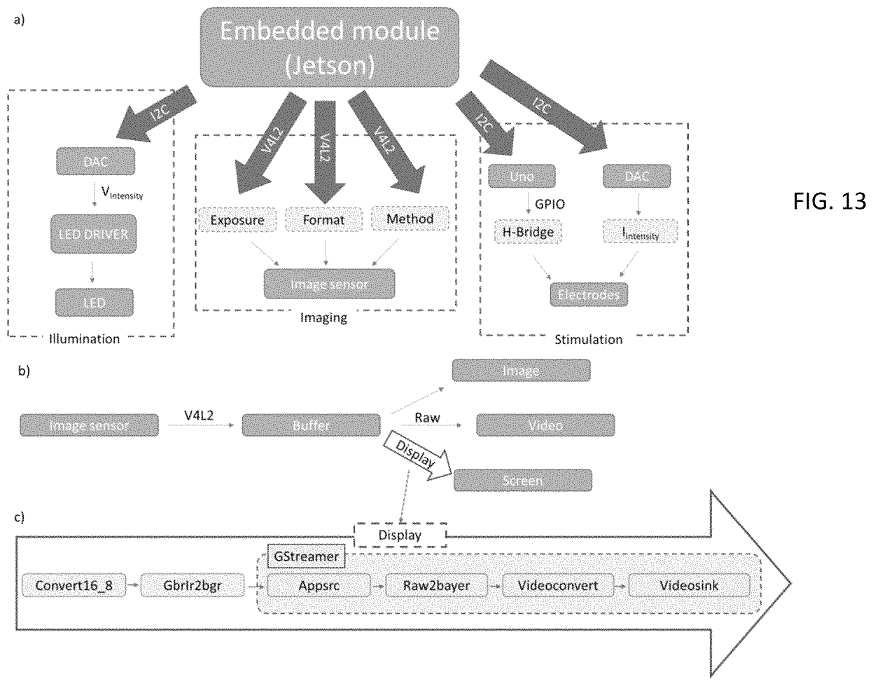

Conditions that alter the development and function of cultured monolayers can affect the spatio-temporal activity. Confluent monolayers usually show consistent and relatively stationary signal with the common contraction/relaxation peaks as shown in FIG. 6, panel A. However, more complex global composite signals can be found such as multiple peak complexes as can be seen in the example shown in FIG. 13, panel A.

In order to investigate the causes of these complex patterns, a thresholding on the energy was done as previously showed (map of log(E) is shown in panel Bi after thresholding with log(E)>4). Calculation of the correlation coefficient with the global composite signals does not highlight important regions with low coefficient (panel Bii). However, calculating the correlation coefficient and lag between cluster signals (from the average ΔS_{x′,y′}(t) of each cluster) results in the matrix plot shown in panel C (correlation coefficient) and D (lag). Both panels highlight a clear difference in correlation and lag between cluster 5 and the others (labelling of the clusters can be found on the map showing the correlation coefficient between cluster 5 and others in panel Biii).

The causes of the correlation differences can be investigated by further studying the differences in the cluster ΔS_{x′,y′}(t) signals represented by a grayscale in panel E. A closer look at the maps shows that synchronisation between cluster 5 and the others varies over time with almost simultaneous activity within the orange dashed rectangle (which has a corresponding normal double peak feature in the global signal shown by the blue line). However, the complex multi-peaks section encompassed within the red dashed rectangle corresponds to an earlier contraction in cluster 5 (with delays between cluster 4 and 5 of 100 ms and 160 ms for the 2^{nd} and 3^{rd} beats in the rectangle). The non-stationary aspect of the global signal can thus be understood by a change in timing of cluster 5 activity in respect to other clusters where the abnormal added peaks are found when a long delay between cluster contractions occurs. Depending on signal quality, the contraction/relaxation analysis approach presented in the first part of this article could be done on cluster signals to extract and compare temporal activity variations.

In this example, we examined approaches to study beating dynamics in cell culture. Based on a simple composite signal calculated as the variation in pixels intensity, two main variables can be determined: the time difference and the ratio between the contraction and relaxation peaks. These new variables could be more interesting to determine toxic effects on cardiomyocytes more importantly regarding heart failure risk. Both variables showed to be dependent on the period of activity. Comparison with simulation results show similarities between simulation and experimental data for the time interval between peaks (<Δ_{R-C}>). Surprisingly, while experimental data show an increasing ratio of amplitude with increasing period of activity, simulation results had a decreasing ratio with increasing period. The mathematical modeling assumed an isotonic contraction (36) while a confluent monolayer should probably be a mixed condition between isometric (on a stiff cell culture substrate) and isotonic on the free top side of the cells.

The proposed approaches are interesting with limited impact on the beating cells except for the illumination duration. Light impact on cellular process can be decreased by limiting exposure and careful selection of wavelength bands to favour contrast but it is believed to be minimal (30). As detailed in example 2, lens free imaging could also be used.

The proposed alternative methods described here that aim to study heterogeneity in contraction signal are interesting as they can estimate cell culture characteristics impossible to study directly with classical methods (30, 37). Here, detection of localized abnormal activity (compared to the global activity) could also be a measure of cell deterioration. Moreover, the change in synchronisation between regions, an important variable that can be link to intercellular coupling and be a factor favoring arrhythmia, can be evaluated. As such, actual application of these approaches and evaluation of their relevance as appropriate biomarker of new drug cardiotoxicity could be of great interest.

### REFERENCES FOR EXAMPLE 1

1. Hazeltine L B, Badur M G, Lian X, Das A, Han W, Palecek S P. Temporal impact of substrate mechanics on differentiation of human embryonic stem cells to cardiomyocytes. Acta biomaterialia. 2014; 10(2):604-12.
2. Pillekamp F, Haustein M, Khalil M, Emmelheinz M, Nazzal R, Adelmann R, et al. Contractile properties of early human embryonic stem cell-derived cardiomyocytes: beta-adrenergic stimulation induces positive chronotropy and lusitropy but not inotropy. Stem cells and development. 2012; 21(12):2111-21.
3. Wendel J S, Ye L, Zhang P, Tranquillo R T, Zhang J. Functional Consequences of a Tissue-Engineered Myocardial Patch for Cardiac Repair in a Rat Infarct Model. Tissue engineering Part A. 2013.
4. Zhang D, Shadrin I Y, Lam J, Xian H Q, Snodgrass H R, Bursac N. Tissue-engineered cardiac patch for advanced functional maturation of human ESC-derived cardiomyocytes. Biomaterials. 2013; 34(23):58β-20.
5. Lu T Y, Lin B, Kim J, Sullivan M, Tobita K, Salama G, et al. Repopulation of decellularized mouse heart with human induced pluripotent stem cell-derived cardiovascular progenitor cells. Nature communications. 2013; 4:2307.
6. Vunjak-Novakovic G, Lui K O, Tandon N, Chien K R. Bioengineering heart muscle: a paradigm for regenerative medicine. Annual review of biomedical engineering. 2011; 13:245-67.
7. Nunes S S, Miklas J W, Liu J, Aschar-Sobbi R, Xiao Y, Zhang B, et al. Biowire: a platform for maturation of human pluripotent stem cell-derived cardiomyocytes. Nature methods. 2013; 10(8):781-7.
8. Navarrete E G, Liang P, Lan F, Sanchez-Freire V, Simmons C, Gong T, et al. Screening drug-induced arrhythmia events using human induced pluripotent stem cell-derived cardiomyocytes and low-impedance microelectrode arrays. Circulation. 2013; 128(11 Suppl 1):53-13.
9. Dick E, Rajamohan D, Ronksley J, Denning C. Evaluating the utility of cardiomyocytes from human pluripotent stem cells for drug screening. Biochemical Society transactions. 2010; 38(4):1037-45.
10. Stevens J L, Baker T K. The future of drug safety testing: expanding the view and narrowing the focus. Drug Discov Today. 2009; 14(3-4):162-7.
11. Ferri N, Siegl P, Corsini A, Herrmann J, Lerman A, Benghozi R. Drug attrition during pre-clinical and clinical development: understanding and managing drug-induced cardiotoxicity. Pharmacol Ther. 2013; 138(3):470-84.
12. Menna P, Salvatorelli E, Minotti G. Cardiotoxicity of antitumor drugs. Chem Res Toxicol. 2008; 21(5):978-89.
13. Schimmel K J, Richel D J, van den Brink R B, Guchelaar H J. Cardiotoxicity of cytotoxic drugs. Cancer Treat Rev. 2004; 30(2):181-91.
14. Yeh E T, Tong A T, Lenihan D J, Yusuf S W, Swafford J, Champion C, et al. Cardiovascular complications of cancer therapy: diagnosis, pathogenesis, and management. Circulation. 2004; 109(25):3122-31.
15. Desai V G, Herman E H, Moland C L, Branham W S, Lewis S M, Davis K J, et al. Development of doxorubicin-induced chronic cardiotoxicity in the B6C3F1 mouse model. Toxicol Appl Pharmacol. 2013; 266(1):109-21.
16. Alderton P M, Gross J, Green M D. Comparative study of doxorubicin, mitoxantrone, and epirubicin in combination with ICRF-187 (ADR-529) in a chronic cardiotoxicity animal model. Cancer Res. 1992; 52(1):194-201.
17. Herman E H, Rahman A, Ferrans V J, Vick J A, Schein P S. Prevention of chronic doxorubicin cardiotoxicity in beagles by liposomal encapsulation. Cancer Res. 1983; 43(11):5427-32.
18. Engler A J, Carag-Krieger C, Johnson C P, Raab M, Tang H Y, Speicher D W, et al. Embryonic cardiomyocytes beat best on a matrix with heart-like elasticity: scar-like rigidity inhibits beating. J Cell Sci. 2008; 121(Pt 22):3794-802.
19. Dorr R T, Bozak K A, Shipp N G, Hendrix M, Alberts D S, Ahmann F. In vitro rat myocyte cardiotoxicity model for antitumor antibiotics using adenosine triphosphate/protein ratios. Cancer Res. 1988; 48(18):5222-7.
20. Shirhatti V, George M, Chenery R, Krishna G. Structural requirements for inducing cardiotoxicity by anthracycline antibiotics: studies with neonatal rat cardiac myocytes in culture. Toxicol Appl Pharmacol. 1986; 84(1):173-91.
21. Boudreau-Beland J, Duverger J E, Petitjean E, Maguy A, Ledoux J, Comtois P. Spatiotemporal stability of neonatal rat cardiomyocyte monolayers spontaneous activity is dependent on the culture substrate. PIoS one. 2015; 10(6):e0127977.
22. Feinberg A W, Alford P W, Jin H, Ripplinger C M, Werdich A A, Sheehy S P, et al. Controlling the contractile strength of engineered cardiac muscle by hierarchal tissue architecture. Biomaterials. 2012; 33(23):5732-41.
23. Sheehy S P, Grosberg A, Parker K K. The contribution of cellular mechanotransduction to cardiomyocyte form and function. Biomech Model Mechanobiol. 2012; 11(8):1227-39.
24. Sham J S, Hatem S N, Morad M. Species differences in the activity of the Na(+)-Ca2+ exchanger in mammalian cardiac myocytes. J Physiol. 1995; 488 (Pt 3):623-31.
25. Zhang J, Wilson G F, Soerens A G, Koonce C H, Yu J, Palecek S P, et al. Functional cardiomyocytes derived from human induced pluripotent stem cells. Circ Res. 2009; 104(4):e30-41.
26. Grosberg A, Alford P W, McCain M L, Parker K K. Ensembles of engineered cardiac tissues for physiological and pharmacological study: heart on a chip. Lab Chip. 2011; 11(24):4165-73.
27. McCain M L, Sheehy S P, Grosberg A, Goss J A, Parker K K. Recapitulating maladaptive, multiscale remodeling of failing myocardium on a chip. Proc Natl Acad Sci USA. 2013; 110(24):9770-5.
28. Chiu L L, Iyer R K, King J P, Radisic M. Biphasic electrical field stimulation aids in tissue engineering of multicell-type cardiac organoids. Tissue Eng Part A. 2011; 17(11-12):1465-77.
29. McCain M L, Agarwal A, Nesmith H W, Nesmith A P, Parker K K. Micromolded gelatin hydrogels for extended culture of engineered cardiac tissues. Biomaterials. 2014; 35(21):5462-71.
30. Rohr S. A computerized device for long-term measurements of the contraction frequency of cultured rat heart cells under stable incubating conditions. Pflugers Arch. 1990; 416(1-2):201-6.
31. Zheng G, Lee S A, Antebi Y, Elowitz M B, Yang C. The ePetri dish, an on-chip cell imaging platform based on subpixel perspective sweeping microscopy (SPSM). Proc Natl Acad Sci USA. 2011; 108(41):16889-94.
32. Korhonen T, Hanninen S L, Tavi P. Model of excitation-contraction coupling of rat neonatal ventricular myocytes. Biophys J. 2009; 96(3):1189-209.
33. Wang L J, Sobie E A. Mathematical model of the neonatal mouse ventricular action potential. Am J Physiol Heart Circ Physiol. 2008; 294(6):H2565-75.
34. Cerbai E, Pino R, Sartiani L, Mugelli A. Influence of postnatal-development on 1(f) occurrence and properties in neonatal rat ventricular myocytes. Cardiovasc Res. 1999; 42(2):416-23.
35. Wickenden A D, Kaprielian R, Parker T G, Jones O T, Backx P H. Effects of development and thyroid hormone on K+ currents and K+ channel gene expression in rat ventricle. J Physiol. 1997; 504 (Pt 2):271-86.
36. Rice J J, Wang F, Bers D M, de Tombe P P. Approximate model of cooperative activation and crossbridge cycling in cardiac muscle using ordinary differential equations. Biophys J. 2008; 95(5):2368-90.
37. Kim S B, Bae H, Cha J M, Moon S J, Dokmeci M R, Cropek D M, et al. A cell-based biosensor for real-time detection of cardiotoxicity using lensfree imaging. Lab Chip. 2011; 11(10):1801-7.
38. Pushkarsky I, Liu Y, Weaver W, Su T W, Mudanyali O, Ozcan A, et al. Automated single-cell motility analysis on a chip using lensfree microscopy. Sci Rep. 2014; 4:4717.

### Example 2

In this example, a lensless real-time system usable to monitor cardiac cell sheet, and other cells, function and structure in culture is describe in further details. This system is an example of implementation of the culture system 300 described hereinabove. This system allows the evaluation of contractile properties as well as structural cell organization at any moment during culture. Additionally, an electrical stimulation unit is provided, allowing for stimulation protocols and long-term stimulation of the tissues.

An implementation of the culture system 300 was developed for real time monitoring and evaluation of tissue cultures. The main processing unit of the system is a Jetson™ TK1 graphic development board, allowing high throughput of frame processing. Using the Jetson TK1 with Ubuntu 14.04 installed, enables the system to behave as a stand-alone device. A screen is connected using a HDMI output, and a wireless mouse and keyboard are connected in the available USB3 port. It is also possible to connect the Jetson to the internet.

The selected imaging sensor 312 is the e-CAM40_CUTK1 (E-Con System™ Inc., India) which was the fastest sensor compatible with the Jetson TK1 board available at the time of development. The camera uses a 4-lane MIPI CSI-2 interface to the board. It comes on a mounting board with its drivers, allowing for easy “plug-and-play” with the Jetson board.

As our system is lensless, the “shadow-imaging” approach was selected as the imaging technique. Its wide field of view and close to none post processing make it a suitable technique for the present implementation. In brief, our light source 314 include, a LED 318 (M505L2, Thorlabs™ Inc.) controlled by a LED driver (DC2100, Thorlabs inc, USA), illuminates a pinhole 320 (D610-1500-MD, Thorlabs inc), which is positioned approximately 5 cm perpendicular to the sample. Since the pinhole is distanced from the sample, the light waves at that distance can be considered planar and the objects, which in our case are mostly transparent, refract light. As the imaging sensor 312 is very close (less than 200 μm) the diffraction is minimal, and the images received consist of small rings corresponding to the area of each cell. The sensor of the imaging sensor 312 is the Omnivision™ OV4682 RGB IR—⅓″ Optical format CMOS which has a pixel size of 2 μm by 2 μm with a surface area of 5.44 mm by 3.07 mm. In order to modulate the intensity of the LED, it is possible to manually control the output of the LED driver or to have it controlled externally by a low voltage input. Our system allows for both, with the modulated voltage input controlled by a digital to analog converter (DAC) (MCP4725, Microchip™) wired to the Jetson TK1 and communicated with by I2C. The gen2-I2C bus is used for communication as it works with 3.3V and is available from the expansion header on pins J3A1.18 and J3A1.20.

A humidity and heat sensor (SEN0137, DFRobot) was also provided.

As mentioned previously, in order to minimize the light diffraction, it is advantageous to minimize the distance between the imaging sensor 312 and the sample. Because of this and because the imaging surface area of our system is relatively small, a custom cell culture well 3-2 is used. In the present invention, the culture well is made with Polydimethylsiloxane (PDMS) (184 Silicone Elastomer Kit, Sylgard), a transparent and widely used silicon-based organic polymer (J. N. Lee et al. 2004). These wells not only have thin bottoms (<100 μm), but also have tunable rigidity by selecting the ratio of curing to polymer. These wells are created with a designed two-part mold. PDMS is first mixed, then stirred for five minutes and degassed before being poured into the mold. Two carbon electrodes 326 (˜4×5×1.25 mm, SK-05 ISO Graphite Plates, Industrial Graphite Sales LLC) can be placed in the mold before curing to permit tissue electrical stimulation. To assure a flat finish and the exact height of the wells' bottom, a plastic cover slip is gently placed over the mold, making sure not to form any bubbles in the process. Once cured, if electrodes have been included, holes are punched on the side of the wells and 1-inch wires are soldered with silver paste (8331-14G, MG Chemicals) to the electrodes. In a specific embodiment, The resulting wells have an outside diameter of 11.9 mm, an inside diameter of 9.0 mm and a height of 4.5 mm, while the primary culture area between the electrodes is of 3.1 mm by 5.5 mm. Before cell culture is performed in these wells, since PDMS is hydrophobic (Bodas and Khan-Malek 2007), a 60 seconds air plasma treatment (PDC-32G, Harrick Plasma Inc) was performed to improve hydrophilicity of the membrane. Plasma treatment of the wells helps with adhesion of the cells.

An electrical stimulation system 306 has been developed for acute cardiac tissue evaluation as well as for chronic stimulation for stem cell-derived cardiomyocyte maturation. This unit has a tunable frequency, duration of pulse and current intensity. The unit function is based on an Arduino Uno driving a “H-Bridge”-like electrical circuit. In brief, stimulation intensity is modulated using a DAC (MCP4725, MicroChip) connected to a non-inverting op-amp circuit.

All functionalities of the system are controllable by the Jetson TK1, which is therefore connected to the imaging sensor 312, the electrical stimulator 324 and the light source 316. The main programs can be divided into four categories: illumination, imaging, electrical stimulation and analysis. The entirety of the code for the embedded system has been written in C, while part of the analysis' post-processing is performed in MATLAB™ (Mathworks, 2018).

The first two functionalities have been grouped and presented in a graphical user interface (GUI) developed with GTK3+(GTK+Team). In brief, to communicate with the DACs, the Linux™ library i2c-dev is used as well as the system ioctl library. The I2C pins are accessed on an adapter from the userspace and the I2C file, once opened, is written to with the “i2c_smbus_write_word_data” function. For the imaging system, the V4L2 API is used to manage the setup and frame grabbing of the imaging sensor, while GStreamer is used for image handling and display. The principal steps for the first part are the following: opening the device, setting up the exposure, negotiate the frame format and the method used to access the frames, create and allocate the memory for the different buffers, generate a main loop where frames are acquired from the sensor and, when finished with the program, closing the device and freeing the memory. Here, the main loop of the code is called by a timer with a duration of 3 ms, the method used is primarily mmap and the frame format is BGGR (BG10) with either 672×380 or 2688×1520 pixels per frame acquired. The theoretical acquisition speed is capped at 330 frames per second (fps) as given by the manufacturer, justifying our use of a timer every 3 ms. It s worth noting that in practice the frame rate is going to be limited by the selected exposure. In the main loop, each grabbed frame is put in a buffer from which three actions can be done simultaneously. If a video is being recorded, each of these frames is temporarily placed in a larger buffer until enough frames have been acquired and the whole buffer is written to a binary file. This method allows for high speed acquisition and conservation of the entire information contained in the frames. To display the grabbed frames on screen, one frame every 60 ms is pushed to the GStreamer pipeline. The format of the frames is specific to the CMOS sensor and takes the 10 bits raw BGR-IR format, meaning instead of a second green pixel it acquires infra-red (IR) intensity and each color pixel is stored in a 16 bits word where the 6 most significant bits are unused. For simplicity and to facilitate the use of pre-existing GStreamer elements, pre-processing of the frames is performed before feeding them to the Appsrc element: Replacing the IR pixel with green values and converting the 16 bits per pixel to 8 bits. To save images to memory, a similar pipeline to the display one has been created with a mpeg and filesink element replacing the videosink element.

In the present example, Image analysis is performed in two steps at this time. The first step, described here, is done while the signal is acquired, whereas the second is done in post processing on a workstation. However, in alternative embodiments, all image processing can be performed offline or in real time. The two follow the same logic to get the contraction rate of the tissue. The idea is to get the light intensity speed of variation per pixel, as captured by the sensor. Since the LED intensity is kept constant, the only thing affecting the intensity received by the sensor is the cells contraction. To calculate the speed of light intensity, we subtract the total frame intensity of two frames and divide by the time separating the two. As the amount of frames per second acquired is known, the only parameter that needs to be set is the distance between frames (τ). In a specific example, this is set at 6 times the fps divided by 100, as based on empirical analysis. For a video with an acquisition speed of 200 fps, this means the delay between frames would be of Σ=12 frames. The equation hereinabove is therefore used to acquire the composite signal ΔS.

This derivative like algorithm gives a continuous signal characterised by two peaks, one of which is the contraction of the tissue and the other the relaxation, as explained in example 1. ΔS curves similar to the curves described with respect to example 1 are obtained using the present setup. The contraction peaks are found automatically using an algorithm which uses an adaptive threshold, first derivative sign change and a minimum peak width. By getting the difference in time between two contraction peaks, we deduce the beating rate of the tissue and display it in real time.

The post processing consists of creating movie format files from the binary files acquired with the camera, getting more information from the composite signal described above and creating activation maps of recorded contractions. The first is either done on the embedded system, by passing the frames read from the binary files to a GStreamer pipeline, or with MATLAB™ on a separate workstation. This GStreamer pipeline has the same elements as in FIG. 13 panel c, but with a video encoder and file sink elements instead of the video sink. In MATLAB, this task is performed using the videowriter object and the raw2rgb functions. In addition to the beating frequency, we propose to look at the ratio between the contraction and the relaxation peak and the time between the two. These signal characteristics are an important source of information as they are directly impacted by physiological phenomenon. For example, if the tissue cells' action potential is increased by the use of lower culture temperatures, the time between the contraction and relaxation peak is expected to increase. To get these signal characteristics, the peaks are found in MATLAB using the “findpeaks” function with custom input arguments and the characteristics computed automatically. The final postprocessing task proposed is the creation of activation maps. These maps allow to determine the propagation speed of the contractile activity and to evaluate the ectopic stability. To achieve this, a window, for example 31 by 31 windows, is first passed over each frame and the contractile signal, the speed of light intensity variation, is computed for each pixel. All contraction peaks are found, and each beat is isolated. Maps are then plotted where the time at which each pixel has contracted is color-displayed.

To test the different functionalities of the presented device, different cardiomyocytes cell lines were used: neonatal rat cardiomyocytes (NRCM), commercial (NCardia-CM) and non-commercial human induced pluripotent stem cell derived cardiomyocytes (hiPS-CM). Each one was cultured according to their respective protocols, as detailed below. The validated functionalities with all three cell types were the capacity to acquire clear images of the cultures, obtain videos of contracting tissue, compute the contractile signal from these videos, which is characterised by their contraction-relaxation pair of peaks, and creating the activation maps. Electrical stimulation validation was performed only with the NRCMs.

Animal handling procedures were conducted according to the Canadian Council on Animal Care guidelines and were approved by the Montreal Heart Institute Animal Research Ethics Committee. Beating hearts were removed from sacrificed Sprague-Dawley rats, aged one to three days old, and kept on ice in Ca²⁺ and Mg²⁺ free Hank's balanced salt solution. Ventricular muscle tissue was then excised and cut into 1-3 mm² pieces. Purification was performed over night using a Neonatal Cardiomyocytes Isolation System (Worthington Biochemical). In parallel, PDMS wells were immersed into 70% ethanol and rinsed with distilled water, plasma-cleaned for 1 minute (PDC-32G, Harrick Plasma Inc) and then sterilized under UV light for 1 hour. Following purification, cells were seeded in the prepared PDMS wells at a density of 30 000 cells per well and maintained in Dulbecco's Modified Eagle's Medium (DMEM, SLM-220-B, Millipore) with 10% fetal bovine serum (FBS, ES-009-B, Millipore) and 1% penicillin/streptomycin (TMS-AB2-C, Millipore). Culture wells containing the cells were placed either on the sensor or in a 10 cm petri dish and in the incubator at 37° C. and 5% CO₂. Imaging was performed 3 days after seeding.

Cor.4U® Human iPS Cell-Derived Cardiomyocytes (NCardia, Germany) were thawed as described in the manufacturer's protocol and maintained in Cor.4U® complete medium as previously described (Gélinas et al. 2017). Briefly, hiPSC-CMs were thawed and plated onto 0.1% gelatin coated 12-well plate (2×105 cells per well) and culture medium was replaced every 3 days until analysis (less than 60 days). hiPSC-CM were then dissociated by incubation with TrypLE Express (Gibco) at 37° C. for 10 minutes before being plated directly onto the gelatin coated PDMS wells (10×10⁴ cells per well). Spontaneously beating cells were seen 2 days after being plated. Imaging was performed two days after observing spontaneous activity.

Commercially available hiPSC (CW200-47, CIRM) were differentiated to hiPSC-CM using a small molecule modulation differentiation protocol followed by a glucose starvation as previous published by Sharma A. et al. (Sharma et al. 2015). Briefly, approximately 100 000 hiPSC were plated on hESC qualified Matrigel (Corning) coated 6-wells culture plate. Once 85% confluency was reached, hiPSC were incubated with 6 μM CHIR99021 (Sigma) in RPMI/B27 minus insulin (Life Technologies) for 48 hr. After 48 hr medium was changed to RPMI/B27 minus insulin until day 3 when medium was replaced by RPMI/B27 minus insulin with 5 μM Wnt inhibitor IWR1 (Sigma) for 48 hr. At day 5 medium was changed to RPMI/B27 minus insulin for 2 additional days. At day 7, medium was replaced with RPMI/B27 (with insulin). Spontaneously beating hiPSC-CMs were observed between day 7 and day 10. At day 10, medium was changed to low glucose RPMI/B27 and cells were maintained in this medium for 3 days. At day 13, cells were washed with DPBS to inhibit contraction and cells were dissociated using TrypLE Express (Gibco) with 0.5 U/ml liberase TH (Roche) at 37° C. for 10 minutes. Cells were then diluted in 5 ml RPMI/B27 before being centrifuged. Cells were resuspended in 1 ml RPMI/B27 and passed through a 100 μm cell strainer before being plated directly onto the Matrigel coated PDMS wells (5×10⁴ cells per well). Contraction was seen after 3 to 5 days.

All cell cultures were performed according to the protocols presented above and in accordance with good manufacturing practices (GMP). For the cardiomyocytes derived from stem cells, the phenotype was deemed achieved when tissue contraction was observed. In neonatal rat and NCardia's cardiomyocytes, close to 100% of the tissue was considered to be cardiomyocytes, while the hiPSC-CM were close to 100% differentiation following purification. An image of cardiomyocytes being cultured in a PDMS well is presented in FIG. 2d.

Each well was unmolded and tested for leaks with dH₂O, then submerged in 70% ethanol before being sterilized under UV light for a minimum of one hour. For neonatal cardiomyocytes, wells were plasma-cleaned before being sterilized by UV.

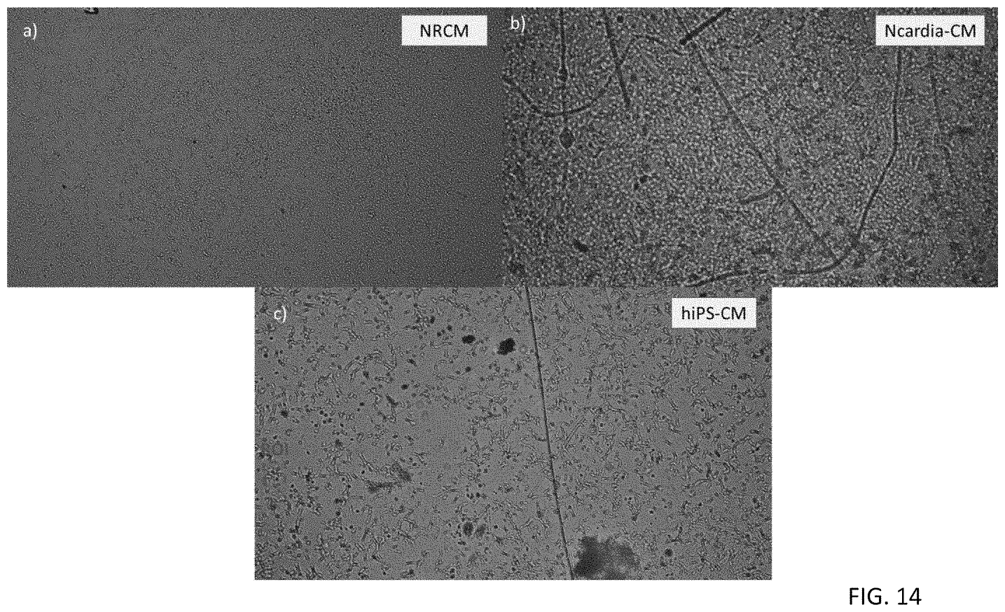

All cell types were imaged using the system. A sample image of each cell type is presented in FIG. 14. For each of these tissue images, exposure was set to 10 ms, the input voltage for light intensity was set to 1.65 V and the frame size was 2668×1520 pixels. In FIG. 14, panel a, neonatal rat cardiomyocytes (NRCM) are imaged three days after seeding in the PDMS well. While it is possible to easily see cell contours, 100% confluency is not achieved in this image as cells would have required additional days to form a fully connected tissue. In FIG. 14, panel b, NCardia's commercially available hiPSC derived cardiomyocytes are imaged after a period of four days after seeding. The long darker lines present in the image are due to scratches and debris on a mold piece. Tissue growth is good in this case as most cells are touching their neighbours. Individual cells are hard to distinguish within the tissue as compared to a lower density. In FIG. 14, panel C, a tissue of cardiomyocytes derived in house from hiPSCs is presented. Seeding was of 5000 cells per well, allowing for clearly distinguishable cells. By doing this we validated the possibility for individual cell monitoring. In summary, these high-resolution images provide knowledge of the tissue organization opening to the possibility for spatial pattern analysis for monitoring.

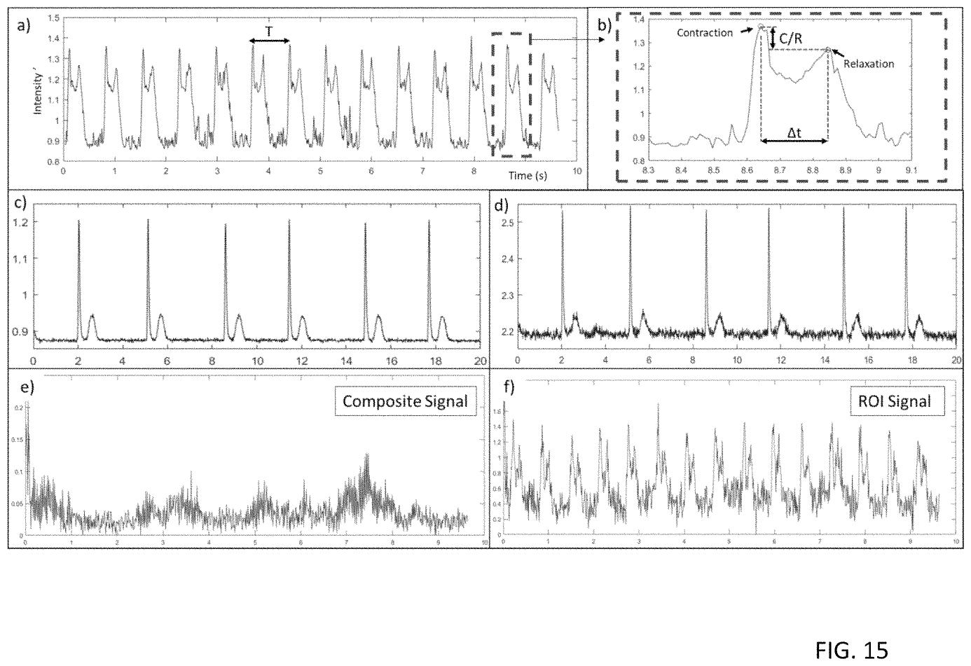

After images of the tissue were taken, video acquisitions of 10 to 20 seconds, with acquisition speed ranging from 100 to 300 fps, were recorded. As these videos were taken, we made sure the illumination of the tissue was appropriate as the exposure of the sensor had to be changed. In addition, contractions could be observed in real-time, confirming the tissues were usable for our analysis purposes and validation. Contractile signals for each cell type obtained from these videos are presented in FIG. 15. In panel a), we show the signal obtained from a tissue of commercial cardiomyocytes derived from hiPSCs. The contraction and relaxation peaks observed are due to the spontaneous activity of the tissue. As an example of the signal characteristics derived from such signal, the average characteristic parameters of this tissue were the following: a period (T) of 710 ms, a time between contraction and relaxation (at) of 200 ms and a contraction intensity over relaxation intensity ratio (C/R) of 0.94. In FIGS. 6c and 6d the signal is obtained from tissue of neonatal rat cardiomyocytes. FIG. 6e shows the signal from a tissue of cardiomyocytes derived from hiPSCs while FIG. 6f shows the signal from mESC derived cardiomyocytes.

The stability of these parameters was evaluated within the same acquisition and over time while keeping the environment stable (data not shown). Four video recordings of 10 seconds of the same NCardia-CM tissue, taken 30 minutes apart, were used for this analysis. It was observed that all three characteristics were stable. Within the same recordings, the highest standard deviation measured was of 0.090 for a period of 1.35 s, 0.026 s for a delay between peaks of 0.340 s and 0.034 s for a peak ratio of 1.243. Over time, the means of each of the average characteristics from each video had standard deviations no higher than 0.01, meaning the measurements were reproducible if the environment is kept stable.

Signals obtained from different wells containing same species tissue were compared in regards of the contractile characteristics dependent on the spontaneous beating period. For this, we used seven different wells containing neonatal rat cardiomyocytes. The seeding density was of 1.9×10⁵ cells per cm². We recorded videos three days after seeding, with each video lasting 10 s with acquisition speeds of 100 fps. Four of these tissues had beating rates higher than 2 Hz, while 6 had rates higher than 1.5 Hz. Only one had a lower beating rate, which was of around 0.33 Hz. No exact relations can be deduced from these measurements, although a trend is observable; delta decrease as the beating rate increases while the peak ratios increases with the frequency.

In panels c and d, we show the impact of the delay between frames, “τ” in equation 1, on the signals obtained from the same video recording of spontaneous activity of neonatal rat cardiomyocytes. In panel c, we used a delay of 6 images (with 100 fps), meaning the time between frames is roughly 60 ms, whereas in FIG. 6d, we used a delay of 3 images, resulting in a 30 ms delay. As it is observable when comparing the two, panel d has a noisier signal which can lead to harder peak detection, as it can be observed in the first relaxation peak. On the other hand, having a smaller delay means the speed of variation of light intensity is closer to the instantaneous change and the obtained signal is more representative of the contraction speed of the tissue.

Activation maps of the different tissue types were produced from recordings as per the methods previously described. Activation maps allow to find the origin of contraction as well as the stability of the ectopic center. Activation maps further allow to characterize conduction in the tissues, including directional conduction velocity and heterogeneity of conduction, for example zone of block. Superimposing the activation map with the image gives more insight into the structural dynamics of the tissue as we can determine where connections are present and which regions contract.

The electrical stimulation sub-unit has been tested separately with the use of an oscilloscope and a 5 W resistance of 15Ω. This setup allowed the confirmation of the biphasic generation of stimulation pulses with tunable frequency, duration of pulse and amplitude, all of which are modified through the main imaging unit. Once tested, cell cultures in wells containing electrodes were connected to the stimulation unit. In FIG. 15, three contractile signals are presented of NRCM tissue, initially without uniform tissue contraction. In panel a, the tissue is connected to the stimulation sub-unit, but no pulses are generated resulting in a noisy signal without definite contraction peaks because the tissue has many areas contracting separately, which results in smaller and varying peaks of the signal. In panels b and c, electrical pulses of 1 Hz and 2 Hz frequency respectively are generated. In these two signals the contraction peaks are identified using the algorithm presented in the methods and the period between beats is computed. In both cases, the period is equal to the stimulating period with a small difference of a few milliseconds.

We proposed a novel imaging system for continuous evaluation of cardiac tissue, integrated with electrical stimulation and modulable culture substrates. This stand-alone device is not only capable of imaging and recording videos of cardiomyocyte cultures, reaching acquisition speeds up to 300 fps, but also create an easily computed characteristic signal of the contraction present in the tissue. The stimulation unit can generate a uniform contraction of the tissue enabling stimulation protocol that could allow further analysis of the tissue as well as long term stimulation protocols for cardiomyocyte maturation after differentiation. Activation maps, providing information on the ectopic origin, contraction propagation speed, and conduction velocity based on delay of contraction between sites, are computed from the contractile signal. This imaging tool combines many important systems to allow a novel approach to evaluate and monitor cardiomyocyte cultures.

One use for this tool, in addition to being a cardiac tissue monitoring and evaluation tool, resides in stem cell differentiation protocol optimization. As an evaluation tool, the characteristic signal gives rapid insight of the contractile properties of the cells. This is then used to evaluate genetic mutations in cardiomyocytes derived from patients or induced mutations by transfection. Currently, methods used such as patch clamp or microelectrode array (MEA) do not allow such quick evaluation nor simplicity. Additionally, quick evaluation of contractile function, in combination with the possibility to run multiple experiments in parallel make this imaging system a useful tool for drug screening. In the optic of optimising cell cultures, real-time imaging systems allow the possibility to extract information, in this case contractile function, at any given time of the culture process. While the cardiac differentiation process is quite slow in comparison to an action potential, the imaging device can be used to follow slower cellular phenomenon, without impacting the cultures environment. To this intent, the device can be programmed to acquire 4 megapixels images at long intervals to follow the development of the tissue and its structural organisation. Once contraction would begin, faster acquisition could be acquired alternately. The contractile properties and the structural information could in turn be used to optimize current protocols or develop new techniques that could complement the current protocols used, such as electrical stimulation during cardiac differentiation.

In the literature, experimental electrical stimulation protocols have already been reported. Long term electrical stimulation, for a period longer than 72 h for example, does not seem to affect cell viability, but enhances cell elongation, preserves contractile function, accelerates cardiomyocyte growth and increases their RNA accumulation (Au et al. 2007; Berger et al. 1994; Johnson et al. 1994).

Cardiomyocyte-like cells derived from stem cells have been shown to achieve a more mature phenotype with the use of a long-term electrical stimulation (Hirt et al. 2014; Chan et al. 2013; Ma et al. 2018). Hernandez et al. have published that even single brief periods of electrical stimulation can promote cardiogenic potential of hiPSCs in terms of the number of beating EBs and gene expression of cardiac transcription factors and contractile muscle proteins (Hernandez et al. 2016). By combining a continuous electrical stimulation with the proposed device, the goal is to allow monitoring of maturing tissues and its process, allowing a better understanding.

The software used in this example may be on a stored on one or more non-transitory computer-readable storage medium/media.

Although the present invention has been described hereinabove by way of exemplary embodiments thereof, it will be readily appreciated that many modifications are possible in the exemplary embodiments without materially departing from the novel teachings and advantages of this invention. Accordingly, the scope of the claims should not be limited by the exemplary embodiments, but should be given the broadest interpretation consistent with the description as a whole. The present invention can thus be modified without departing from the spirit and nature of the subject invention as defined in the appended claims.

What is claimed is:

1. A characterization method for characterizing contractions of a tissue in vitro, the tissue including cardiomyocytes, the method comprising
capturing a sequence of images of at least part of the tissue over a predetermined time period, each of the images including a plurality of pixels, each pixel being characterized by a respective intensity value; and
for each pair of images from the sequence of images separated by a predetermined delay, computing a movement index over a common region of the images, computing the movement index including summing over all the pixels contained in the common region an absolute value of a difference between the intensity values of the pixels in the images of the pair of images, the movement indexes forming a movement index sequence;
wherein the movement index sequence is indicative of changes in time of sarcomere length in the cardiomyocytes contained in the region.

2. The characterization method as defined in claim 1, wherein characterizing the contractions of the tissue includes detecting peaks in the movement index sequence.

3. The characterization method as defined in claim 2, wherein the peaks include alternating major peaks and minor peaks, characterizing the contractions of the tissue includes calculating an amplitude ratio between major and minor peaks.

4. The characterization method as defined claim 2, wherein characterizing the contractions of the tissue includes calculating a peak delay between at least two of the peaks.

5. The characterization method as defined in claim 1, further comprising culturing the tissue in a monolayer prior to capturing the sequence of images.

6. The characterization method as defined in claim 1, wherein the movement index is a global movement index and the movement index sequence is a global movement index sequence, the method further comprising computing local movement indexes in a sub-region of the common region to obtain a local movement index sequence, characterizing the contractions of the tissue including comparing the local and global movement index sequences.

7. The characterization method as defined in claim 6, wherein comparing the local and global movement index sequences includes computing a delay between the peaks of the local movement index sequence and the peaks of the global movement index sequence.

8. The characterization method as defined in claim 1, wherein capturing the sequence of images is performed using lensless imaging.

9. An assessment method for assessing an effect of a stimulus on cardiomyocytes contraction, the cardiomyocytes being part of an in vitro tissue sample, the assessment method comprising:
applying the stimulus to the in vitro tissue sample;
obtaining a post-stimulus movement index sequence associated with the tissue after application of the stimulus using the method of claim 1;
assessing the effect of the stimulus on the cardiomyocytes by comparing the post-stimulus movement index sequence to a pre-stimulus movement index sequence obtained either using the method of claim 1 on the in vitro tissue sample prior to application of the stimulus or obtained using a reference tissue sample similar to the in vitro tissue sample.

10. The assessment method as defined in claim 9, wherein the stimulus is selected from the group consisting of pharmacological, chemical, physical, mechanical and electrical stimuli.

11. The assessment method as defined in claim 9, wherein assessing the effect of the stimulus includes comparing contraction periods obtained from the post-stimulus movement index sequence and from the pre-stimulus movement index sequence.

12. The assessment method as defined in claim 9, wherein assessing the effect of the stimulus includes comparing contraction amplitudes obtained from the post-stimulus movement index sequence and from the pre-stimulus movement index sequence.

13. The assessment method as defined in claim 9, wherein assessing the effect of the stimulus includes comparing a variation in delay between successive contractions in the post-stimulus movement index sequence and in the pre-stimulus movement index sequence.

14. The assessment method as defined in claim 9, wherein assessing the effect of the stimulus includes comparing root mean square values of the post-stimulus and pre-stimulus movement index sequences.

15. An apparatus for analyzing contractions of a tissue sample including cardiomyocytes on basis of a sequence of captured images, the apparatus comprising
an image acquisition system configured to obtain the sequence of images depicting the tissue sample over an analysis period;
an analysis system configured to compute the movement index sequence as defined in claim 1.

16. The apparatus as defined in claim 15, further comprising a culture chamber for containing the tissue sample.

17. The apparatus as defined in claim 16, wherein the culture chamber is provided with a window, and the image acquisition portion is provided with an image sensor in register with the window and configured to perform lensless imaging of the tissue sample.

18. The apparatus as defined in claim 17, further comprising at least one electrode for applying an electrical stimulus to the tissue sample.
